(19) United States
(12) Patent Application Publication
(10) Pub. No.: US 2011/0071072 A1
(43) Pub. Date: Mar. 24, 2011

(54) Antifungal Bone Cements

(75) Inventors: Richard A. Calderone, Chevy Chase, MD (US); Michael O'Reilly, La Plata, MD (US)

(73) Assignee: Georgetown University, A Congressionally Chartered Institution of Higher Education, Washington, DC (US)

(21) Appl. No.: 12/869,366

(22) Filed: Aug. 26, 2010

Related U.S. Application Data
(60) Provisional application No. 61/237,973, filed on Aug. 28, 2009.

(51) Int. Cl. A61K 38/12; A61P 41/00; A61P 31/10

(52) U.S. Cl. 514/3.4; 514/3.3

(57) ABSTRACT

Antifungal bone cement compositions and methods of using the same are disclosed. In one aspect, a bone cement composition comprises an echinocandin lipopeptide antifungal agent and a cementing agent. Optionally, the echinocandin lipopeptide antifungal agent is selected from the group consisting of micafungin, caspofungin and anidulafungin. The echinocandin lipopeptide antifungal agent is optionally micafungin. Methods of preventing a fungal infection following an arthroplasty procedure are also provided.

### CROSS-REFERENCE TO RELATED APPLICATIONS

This application claims the benefit of U.S. Provisional Patent Application No. 61/237,973, filed Aug. 28, 2009, which is incorporated herein by reference in its entirety.

### STATEMENT REGARDING FEDERALLY FUNDED RESEARCH

This invention was made with government support under Grant NIAID 43465 awarded by The National Institutes of Health. The government has certain rights in the invention.

### BACKGROUND

Arthroplasty procedures can be complicated by bacterial or fungal infection. Outcomes resulting from fungal infections are often worse than the outcomes of bacterial infections.

### SUMMARY

Antifungal bone cements, compositions and methods of using the same are disclosed. In one aspect, a bone cement composition comprises an echinocandin lipopeptide antifungal agent and a cementing agent. Optionally, the echinocandin lipopeptide antifungal agent is selected from the group consisting of micafungin, caspofungin and anidulafungin. The echinocandin lipopeptide antifungal agent is optionally micafungin.

The cementing agent of the bone cement composition can comprise polymethylmethacrylate and/or methylmethacrylate. The cementing agent can be infiltrated in the bone cement composition with the echinocandin lipopeptide antifungal agent. The bone cement composition can further comprise a liquid component. For example, the liquid component can be a liquid monomer.

Also provided is a composition comprising polymethylmethacrylate or methylmethacrylate infiltrated with an echinocandin lipopeptide antifungal agent. The composition can be a bone cement. Optionally, the echinocandin lipopeptide antifungal agent is selected from the group consisting of micafungin, caspofungin and anidulafungin. The echinocandin lipopeptide antifungal agent is optionally micafungin.

Further provided is a kit for producing bone cement. The kit comprises a bone cementing agent component wherein the bone cementing agent component comprises an echinocandin lipopeptide antifungal agent. Optionally, the bone cementing agent component comprises polymethylmethacrylate and/or methylmethacrylate. The kit can further comprise a bone cement liquid component. The liquid component is optionally a liquid monomer. The cementing agent component and the liquid component can be combined to form a bone cement. The echinocandin lipopeptide antifungal agent is optionally selected from the group consisting of micafungin, caspofungin and anidulafungin. Optionally, the echinocandin lipopeptide antifungal agent is micafungin.

Further provided is a method of preventing a fungal infection following an arthroplasty procedure comprising providing a prosthetic device, providing bone cement comprising an echinocandin lipopeptide antifungal agent and a cementing agent and fixing the prosthetic device to bone using the bone cement. Optionally, the method comprises an antifungal agent that treats a fungal infection caused by a Candida species.

### DESCRIPTION OF DRAWINGS

FIG. 1 shows photographs of Petri dishes illustrating zones of growth inhibition of C. albicans in the presence of cement disks impregnated with voriconazole or echinocandin micafungin. Agar diffusion of micafungin (PMMIC 1-6) or voriconazole (PMVOR1) from PM-cement discs showing the inhibition of growth of C. albicans are shown. The zone of inhibition is much greater with micafungin.

FIG. 2 is a graph showing comparisons of micafungin (MIC) and anidulafungin (ANA) infiltrated in hydrozyapatite (HA) or polymethylmethacrylate (PM)-cement and their inhibitory activity against C. albicans.

### DETAILED DESCRIPTION

Provided herein are antifungal bone cement compositions and methods of using the same. Bone cement compositions comprise cementing agents and can be used to deliver echinocandin lipopeptide antifungal agents to a subject's tissues. A bone cement composition can be produced by mixing a cementing agent component comprising a cementing agent with a liquid component. The cementing agent component can be a powder or a liquid comprising the cementing agent. The cementing agent component can be mixed with the liquid component to form a bone cement. When the cementing agent component and the liquid component are mixed to form the cement, the liquid component can activate catalysts in the cementing agent component causing polymerization. The cement can progresses from a pliable, mixable consistency after the components are mixed to a hardened cement used to fix a prosthesis to bone.

Thus, the polymerizing and polymerized bone cement can be used for seating and fixation of prostheses to bones. In one aspect, a predetermined quantity of a cementing agent component is mixed with a predetermined quantity of a liquid component. Such quantities can be determined based on the particular cementing agent component and liquid component used, and on other desired characteristics such as setting time and clinical application.

To make a cement, the liquid component can be poured into a sterile container to which the cementing agent component can be added. Alternatively, the cementing agent component can be poured into a sterile container to which the liquid component can be added.

The resulting mixture, the bone cement, can be stirred and/or kneaded until desired characteristics are achieved. For example, depending on the clinical application, desired characteristics can relate to the consistency of the cement or the amount of air bubbles in the cement. Kneading can remove air bubbles while maintaining the cement in a pliable state for seating and fixing of a prosthesis to bone. These characteristics can be determined by a medical practitioner or other individual with experience in the use of bone cements. The prosthesis can optionally be held securely in place until the cement has hardened. Excess applied cement can be removed before or after hardening.

The described bone cements comprise an echinocandin lipopeptide antifungal agent and a cementing agent. Echinocandin lipopeptide antifungals inhibit the synthesis of cell wall β-1,3 glucans of most human pathogenic fungi and include caspofungin, micafungin, and anidulafungin. All are therapeutically safe and effective drugs in the treatment of fungal infections such as candidiasis and invasive aspergillosis. Optionally, the echinocandin lipopeptide antifungal agent is selected from the group consisting of micafungin, caspofungin and anidulafungin.

Bone cements are effective for drug delivery when infiltrated with an echinocandin lipopeptide antifungal agent. Bone cements have pores from which an antifungal agent diffuses. The rate of diffusion of an echinocandin lipopeptide antifungal agent from the bone cement can depend on the pore size of the cement used and the properties of the echinocandin lipopeptide antifungal agent used. The amount of echinocandin lipopeptide antifungal agent infiltrated per unit volume of bone cement or per weight of bone cementing agent or of cementing agent component can therefore be adjusted based on, for example, the particular echinocandin lipopeptide antifungal agent used, the particular cement used, and the like. It can also be adjusted depending, for example, on clinical factors such as, but not limited to, the severity of the fungal infection, and the location and type of procedure. PMMA or PM (both abbreviations for polymethylmethacrylate) bone cements are effective cements for delivery of echinocandin lipopeptide antifungal agents when impregnated with the echinocandin lipopeptide antifungal agent.

Some bone cementing agent components or bone cements that comprise cementing agents that can be infiltrated with an echinocandin lipopeptide antifungal agent include, but are not limited to, those from Stryker (Kalamazoo, Mich.), Zimmer (Warsaw, Ind.), DePuy (Warsaw, Ind.), Biomet (Warsaw, Ind.), and Smith & Nephew (Memphis, Tenn.). For example, an echinocandin lipopeptide antifungal agent can be mixed under sterile conditions with individual batches of Depuy (Wardaw, Ind.) Smartset HV®, Polymethymethacrylated bone cement or bone cement cementing agent component.

The cementing agent component can comprise a cementing agent such as polymethylmethacrylate and/or methylmethacrylate. The cementing agent component can be infiltrated with the echinocandin lipopeptide antifungal agent. Optionally, the bone cement can comprise one or more other antimicrobial agents such as antibacterial agents like Gentamicin sulfate. Additionally, the bone cement can comprise radiopaque compositions or compositions for providing desired setting and handling characteristics. For example, the bone cement can comprise methyl-methacrylate-styrene copolymer, polymethylmethacrylate, barium sulfate, benzoyl peroxide, methylmethacrylate-methylacrylate copolymer, methylmethacrylate homopolymer, zirconium dioxide, and chlorophyll. Such compositions can be mixed with the cementing agent component for combination with a liquid component. The liquid component for mixing with the powder component can be a liquid monomer. Optionally, the liquid component can be mixed with a bone cementing agent component comprising the cementing agent and the echinocandin lipopeptide antifungal agent to form a bone cement. Optionally, the liquid component comprises methylmethacrylate and N,N-dimethyl-p-toluidine.

Also provided are compositions comprising polymethylmethacrylate or methylmethacrylate infiltrated with an echinocandin lipopeptide antifungal agent. The composition can be a bone cement. Optionally, the echinocandin lipopeptide antifungal agent is selected from the group consisting of micafungin, caspofungin and anidulafungin.

Further provided are kits for producing bone cement. A kit can comprise a bone cementing agent component comprising an echinocandin lipopeptide antifungal agent. Optionally, the bone cementing agent component comprises polymethylmethacrylate and/or methylmethacrylate. The cementing agent component can comprise a powder or a liquid comprising a cementing agent. The kit can further comprise a bone cement liquid component. The liquid component is optionally a liquid monomer. The bone cementing agent component and the liquid component can be separately packaged prior to use and, as described above, the cementing agent component (including a cementing agent and an antifungal agent) and the liquid component can be combined to form a bone cement. The echinocandin lipopeptide antifungal agent of the kit is optionally selected from the group consisting of micafungin, caspofungin and anidulafungin. Optionally, the echinocandin lipopeptide antifungal agent of the kit is micafungin.

The bone cements, compositions and kits described can be used in all clinical applications where a bone cement is indicated. For example, the bone cements can be used to fix a prosthesis to bone in a patient having, suspected of having, or at risk of developing a fungal infection. Such applications include arthroplasty procedures of the hip, knee, elbow and other anatomical locations. For example, a total joint arthroplasty can be performed by removal of an infected implant, placement of an echinocandin lipopeptide antifungal agent impregnated cement spacer and reimplantation following treatment.

Further provided is a method of preventing a fungal infection following an arthroplasty procedure comprising providing a prosthetic device, providing bone cement comprising an echinocandin lipopeptide antifungal agent and a cementing agent and fixing the prosthetic device to bone using the bone cement. Optionally, the antifungal agent treats or prevents a Candida infection. For example, the Candida species can be selected from the group consisting of C. albicans, C. glabrata, C. parapsilosis and C. krusei.

### Example 1

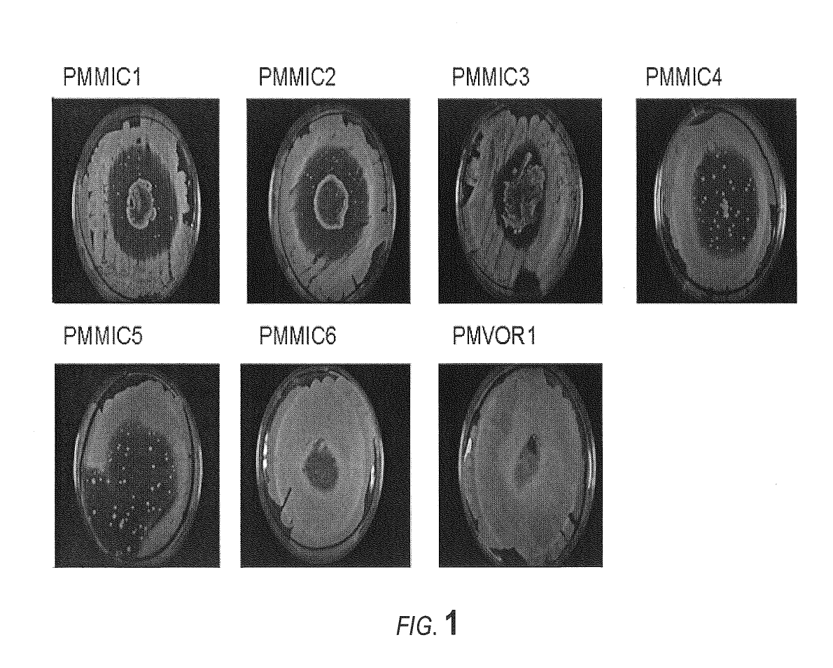

The echinocandin antifungal micafungin or the triazole voriconazole were mixed with polymethylmethacrylate and a bone cement was formed using the mixture. C. albicans was grown overnight in T-soy broth, washed in buffer, then 1000 C. albicans yeast cells were added to the agar surface of a medium containing RPMI, MOPS, and 2% glucose, which was prepared by adding the latter to autoclaved agar after the agar had cooled for 15 min. Cement discs containing micafungin (100 mg) or voriconazole (400 mg) were prepared as described below. Each cement-antifungal disc was placed on the agar culture medium containing the organism. The zones of inhibition surrounding each cement disc, as shown in FIG. 1, were measured daily for 6 days.

The antifungal-cement mix (Smartset HV®, bone cement, DePuy (Warsaw, Ind.)) was prepared as follows under sterile conditions: the polymethylmethacrylate (PM) cement powder (part 1) was first mixed thoroughly with each drug and, then 18.88 g of bone cement liquid (part 2) was added to the mixture. Discs of cement-drug were prepared quickly before the cement hardened. Each cement disc (14 mm) was then placed in the center of inoculated agar cultures in 60 mm petri dishes. The plates were then incubated at 30° C. and zones of inhibition were measured every 24 hrs until activity was not observed (usually 6 days). The same discs were removed daily and placed on fresh agar medium that was inoculated similarly.

The zone of inhibition of growth of C. albicans in the presence of cement discs impregnated with voriconazole or the echinocandin micafungin was measured every 24 hrs until anti-candida activity was no longer detected. At 24 hrs, cement discs containing the antifungal micafungin were about 2.5-fold greater in activity that voriconazole. Further, the activity of micafungin was still detected at 120 hrs post-incubation, while no activity was detected after 24 hrs with voriconazole. Data were plotted as mm growth inhibition versus days of incubation (Table 1). The plates were digitally photographed using image processing software.

TABLE 1

| | 24 | 48 | | | | |
|---|---|---|---|---|---|---|
| Plate | hours | hours | 72 hours | 96 hours | 120 hours | 144 hours |
| PMMIC1 | 33 | 33 | 33 | 38 | 28 | 18 |
| PMMIC2 | 33 | 33 | 33 | 38 | 28 | 20 |
| PMMIC3 | 34 | 30 | 34 | 34 | 30 | 17 |
| PMVOR1 | 14 | | | | | |
| PMVOR2 | 14 | | | | | |
| PMVOR3 | 14 | | | | | |

Table 1 shows results for PM cement prepared with either micafungin (PMMIC1-3) or voriconazole (PMVOR 1-3). The numbers 1-3 indicated triplicate cultures. The zone of inhibition around each disc (in mm) was indicated for each time point. Micafungin was superior and longer lasting in its inhibitory potential compared to voriconazole. Thus, voriconazole was active only for 1 day while micafungin was still active after 6 days of incubation.

### Example 2

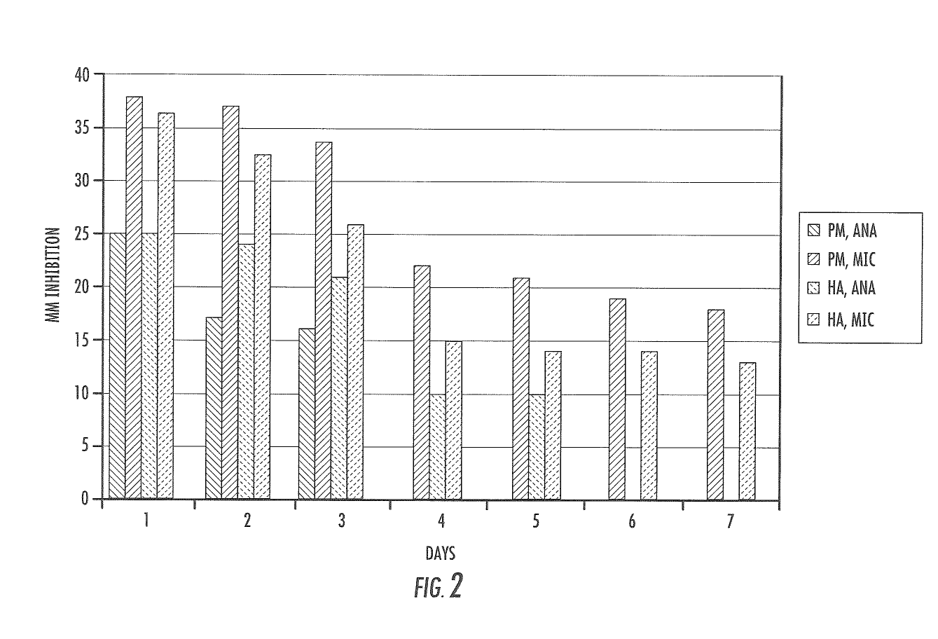

Both PM-cement and hydroxyapatite (HA) infiltrated with either micafungin or anidulafungin were evaluated. As shown in FIG. 2, the activity of each drug was given as millimeters (mm) of growth inhibition. The results again indicated the superiority of micafungin (especially the longevity or diffusion of the drug) regardless of whether the drug was infiltrated in HA or PM cement. The in vitro anti-Candida albicans activity of micafungin was significantly greater than that of voriconazole and anidulafungin. Also, the activity of micafungin persisted after 6-7 days, whereas with voriconzole (and to a lesser extent with anidulafungin), activity was not evident after 1 day.

A number of antifungal compositions, antifungal agents containing bone cements and methods of use have been described. Nevertheless, it will be understood that various modifications may be made without departing from the spirit and scope of the invention.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these combinations may not be explicitly disclosed, each is specifically contemplated and described herein. For example, if a particular modification of a bone cement is disclosed and discussed and a number of modifications that can be made to the bone cement are discussed, each and every combination and permutation of the bone cement are specifically contemplated unless specifically indicated to the contrary. Likewise, any subset or combination of these is also specifically contemplated and disclosed. Similarly, where methods are disclosed to contain specific steps, combinations or subsets of these steps are contemplated herein.

What is claimed is:

1. A bone cement composition comprising an echinocandin lipopeptide antifungal agent and a cementing agent.

2. The bone cement composition of claim 1, wherein the echinocandin lipopeptide antifungal agent is selected from the group consisting of micafungin, caspofungin and anidulafungin.

3. The bone cement composition of claim 2, wherein the echinocandin lipopeptide antifungal agent is micafungin.

4. The bone cement composition of claim 1, wherein the cementing agent comprises polymethylmethacrylate.

5. The bone cement composition of claim 1, wherein the cementing agent comprises methylmethacrylate.

6. The bone cement composition of claim 1, wherein the cementing agent is infiltrated with the echinocandin lipopeptide antifungal agent in the bone cement composition.

7. The bone cement composition of claim 1, wherein the bone cement further comprises a liquid component.

8. The bone cement composition of claim 7, wherein the liquid component is a liquid monomer.

9. A composition comprising polymethylmethacrylate or methylmethacrylate infiltrated with an echinocandin lipopeptide antifungal agent.

10. The composition of claim 9, wherein the echinocandin lipopeptide antifungal agent is selected from the group consisting of micafungin, caspofungin and anidulafungin.

11. The composition of claim 10, wherein the echinocandin lipopeptide antifungal agent is micafungin.

12. A kit for producing bone cement, the kit comprising a bone cementing agent component wherein the bone cementing agent component comprises an echinocandin lipopeptide antifungal agent.

13. The kit of claim 12, further comprising a bone cement liquid component.

14. The kit of claim 13, wherein the liquid component is a liquid monomer.

15. The kit of claim 12, wherein the echinocandin lipopeptide antifungal agent is selected from the group consisting of micafungin, caspofungin and anidulafungin.

16. The kit of claim 15, wherein the echinocandin lipopeptide antifungal agent is micafungin.

17. The kit of claim 12, wherein the bone cementing agent component further comprises polymethylmethacrylate.

18. The kit of claim 12, wherein the bone cementing agent component further comprises methylmethacrylate.

19. A method of preventing a fungal infection following an arthroplasty procedure comprising:
(a) providing a prosthetic device;
(b) providing bone cement comprising an echinocandin lipopeptide antifungal agent and a cementing agent; and
(c) fixing the prosthetic device to bone using the bone cement.

20. The method of claim 19, wherein the antifungal agent treats a Candida species.
